(19) United States
(12) Patent Application Publication
(10) Pub. No.: US 2013/0345545 A1
(43) Pub. Date: Dec. 26, 2013

(54) Ultrasound Enhanced Magnetic Resonance Imaging

(71) Applicants: Patrick Gross, Buckenhof (DE); Chi-Yin Lee, Sammamish, WA (US); Stephen R. Barnes, Bellevue, WA (US); Liexiang Fan, Sammamish, WA (US); Caroline Maleke, Bellevue, WA (US); Kevin Michael Sekins, Yarrow Point, WA (US)

(72) Inventors: Patrick Gross, Buckenhof (DE); Chi-Yin Lee, Sammamish, WA (US); Stephen R. Barnes, Bellevue, WA (US); Liexiang Fan, Sammamish, WA (US); Caroline Maleke, Bellevue, WA (US); Kevin Michael Sekins, Yarrow Point, WA (US)

(73) Assignees: Siemens Medical Solutions USA, Inc., Malvern, PA (US); Siemens Aktiengesellschaft, Munich (DE)

(21) Appl. No.: 13/529,746

(22) Filed: Jun. 21, 2012

(51) Int. Cl. A61B 8/13; A61B 5/055

(52) U.S. Cl. 600/411

(57) ABSTRACT

Magnetic resonance imaging frame rate is increased using ultrasound information. Magnetic resonance (MR) images may be provided at an increased frame rate relative to the MR acquisition. For times between acquisition of MR data, MR data may be created. To account for any change in position of tissue over time, ultrasound is used to track the location of tissue or other imaged structure. The ultrasound-based location information is used to indicate the position of intensities or values of the created MR data. MR images at a higher frame rate than the MR acquisition are generated, but with accuracy of relative position based on the ultrasound data.

### BACKGROUND

The present embodiments relate to magnetic resonance imaging (MRI). In particular, MRI is enhanced with ultrasound.

Both MRI and ultrasound imaging generate images of anatomy. MRI has the benefits of generating clear and crisp images (e.g., higher signal-to-noise ratio) at a high spatial resolution and is less affected by occlusion. However, the acquisition time for even anatomical MRI is slower than ultrasound imaging. Ultrasound imaging may provide real time imaging, even of a volume, at a higher rate, allowing viewing of blood flow and movement of internal organs. Ultrasound imaging may be relatively inexpensive as well. However, ultrasound imaging suffers from speckle, resulting in lower signal-to-noise ratio.

### BRIEF SUMMARY

By way of introduction, the preferred embodiments described below include methods, systems, instructions, and computer readable media for ultrasound enhanced magnetic resonance imaging. Magnetic resonance (MR) images may be provided at an increased frame rate relative to the MR acquisition. For times between acquisition of MR data, MR data may be created. To account for any change in position of tissue over time, ultrasound is used to track the location of tissue or other imaged structure. The ultrasound-based location information is used to indicate the position of intensities or values of the created MR data. MR images at a higher frame rate than the MR acquisition are generated, but with accuracy of relative position based on the ultrasound data.

In a first aspect, a method is provided for ultrasound enhanced magnetic resonance imaging. Ultrasound data representing a region of a patient is acquired at a first rate. A first frame of the ultrasound data for the region is acquired at a first time, and a second frame of the ultrasound data for the region is acquired at a second time after the first time. Scan magnetic resonance data representing the region of the patient is acquired at a second rate less than the first rate. A first frame of the scan magnetic resonance data for the region is acquired at substantially the first time, and a second frame of the scan magnetic resonance data for the region is acquired at a third time after the first and second times. Motion is determined from the first time to the second time from the first and second frames of the ultrasound data. A third frame of constructed magnetic resonance data is constructed from the first frame of the scan magnetic resonance data and the motion. The third frame represents the region at the second time. An image is displayed as a function of the third frame of the constructed magnetic resonance data.

In a second aspect, a non-transitory computer readable storage medium has stored therein data representing instructions executable by a programmed processor for ultrasound enhanced magnetic resonance imaging. The storage medium includes instructions for generating, by magnetic resonance scanning, a temporal sequence comprising sets of first magnetic resonance data, determining, with ultrasound, a change in location over time for each of a plurality of locations represented in at least a first set of the sets of magnetic resonance data, creating additional sets of magnetic resonance data from one or more of the sets of first magnetic resonance data as a function of the changes in locations, and inserting the additional sets into the temporal sequence such that the temporal sequence of the sets and additional sets has a greater frame rate than the sets generated by magnetic resonance scanning.

In a third aspect, a system is provided for ultrasound enhanced magnetic resonance imaging. A magnetic resonance (MR) system is configured to provide a first sequence of frames or volumes of MR data. An ultrasound system is configured to provide a second sequence of frames or volumes of ultrasound data. A processor is configured to determine spatial offsets over time from the ultrasound data and to increase a frame rate of MR images applying the spatial offsets to the MR data.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for ultrasound enhanced magnetic resonance imaging;

FIG. 2 illustrates example acquisition rates and corresponding frames of MR and ultrasound data;

FIG. 3 illustrates a sequence of MR frames, including inserted frames, at a higher frame rate than the acquisition shown in FIG. 2; and

FIG. 4 is a block diagram of one embodiment of a system for ultrasound enhanced magnetic resonance imaging.

### DETAILED DESCRIPTION OF THE DRAWINGS AND PRESENTLY PREFERRED EMBODIMENTS

High temporal resolution MRI is generated with the help of real time ultrasound scanning. A combination of MRI and ultrasound imaging is used to generate MR images with better temporal resolution. Intermediate MR images are generated, given low temporal resolution MR images, with the help of high frame rate ultrasound images. The MRI values may be associated on a voxel-by-voxel or pixel-by-pixel basis with ultrasound values. The motion recorded within ultrasound scanning may be used to reconstruct higher temporal resolution MR images based on a motion model and acquired MRI values. The resulting MR images may capture the motion of anatomical structure.

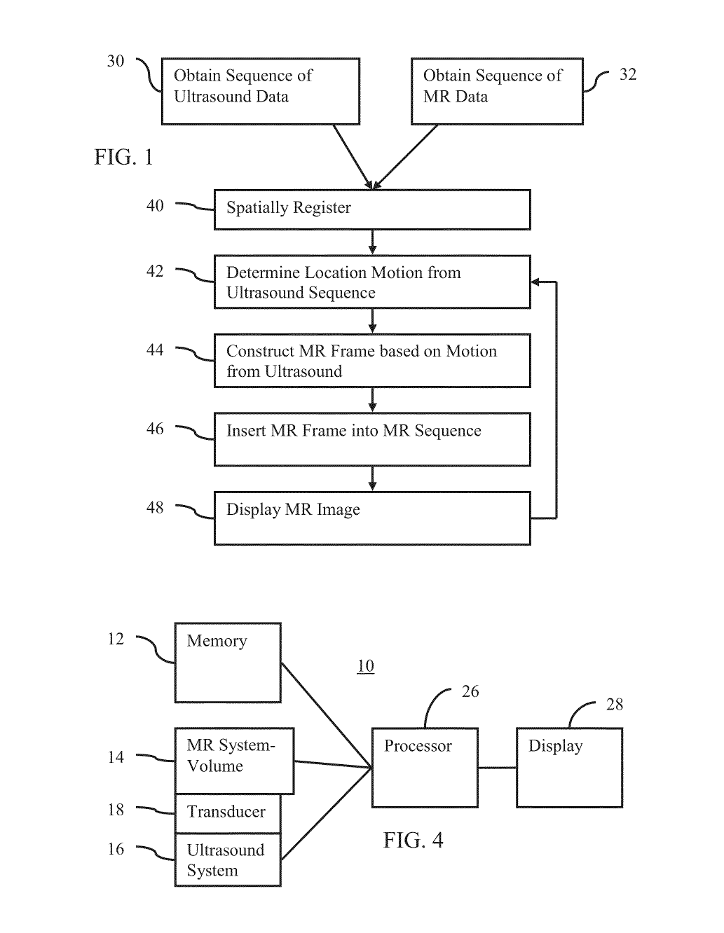

FIG. 1 shows a method for ultrasound enhanced magnetic resonance imaging. The method is implemented by the system 10 of FIG. 4 or another system. The acts are performed in the order shown or other orders. For example, acts 30 and 32 are performed in an interleaved manner, sequentially, or at a same time. For sequentially, an additional act for synchronizing the acquisitions with a cycle, such as heart or breathing cycle, may be performed. Acts 40-48 are performed in real-time with the acquisitions of acts 30 and 32, such as being performed in a same examination session, while acquisition or scanning is occurring, and/or within seconds of having scanned. Alternatively, acts 40-48 are performed at a later time or after an examination session.

Additional, different, or fewer acts may be provided. For example, the spatially registering act 40 is not provided. As another example, act 48 is not provided. In another example, an act for temporally aligning MR and ultrasound data acquired at different times but a same or similar phase of a heart, breathing, or other cycle is provided. The temporal alignment based on physiological cycle provides data representing a same time relative to the cycle.

The acquisitions of acts 30 and 32 are performed by ultrasound and MRI systems. The data is obtained in real-time or during the scans. Alternatively, the data was previously acquired and is obtained by data transfer or access to memory.

In act 30, ultrasound data is acquired. Ultrasound data is acquired by acoustically scanning the patient in two or three dimensions. Any type of scan, scan format, or imaging mode may be used. For example, harmonic imaging is used with or without added contrast agents. As another example, B-mode, color flow mode, spectral Doppler mode, M-mode, contrast, or other imaging mode is used.

Ultrasound data representing anatomical or flow information is acquired from the patient by scanning. The data represents a point, a line, an area, or a volume of the patient. For ultrasound imaging, waveforms at ultrasound frequencies are transmitted, and echoes are received. The acoustic echoes are converted into electrical signals and beamformed to represent sampled locations within a region of the patient. The beamformed data may be filtered or otherwise processed, such as isolating information from a harmonic or fundamental frequency band. Echoes at one or more harmonics of the transmitted waveforms may be processed.

The beamformed data may be detected, such as determining intensity (B-mode) or velocity (flow mode). A sequence of echo signals from a same location may be used to estimate velocity, variance, and/or energy. A sequence may also be used for detecting contrast agents. For example, the response to transmissions with different phases and/or amplitudes is added to isolate information from contrast agents as opposed to tissue or flow. Contrast agent detection may be used for perfusion analysis. Other detection techniques from the beamformed data may be used. The detected ultrasound information is anatomical data. For example, B-mode data represents tissue structures. As another example, flow data indicates locations associated with a vessel. In yet another example, contrast agent data indicates contrast agents associated with structure within the patient.

The detected values may be filtered and/or scan converted to a display format. The ultrasound data representing the patient is from any point along the ultrasound processing path, such as channel data prior to beamformation, radio frequency or in-phase and quadrature data prior to detection, detected data, or scan converted data.

Each scan of a region provides a frame of data. The data of the frame may represent an entire region of interest or field of view, such as an area or volume. The data of each frame is associated with a given time. While ultrasound data of a frame may be acquired at different times (e.g., data for the last scanned location is acquired after data for the first scanned location of a region) or from multiple, sequential transmissions to a same location (e.g., flow ensemble), the data of the frame is acquired to represent the region at a given time relative to a frame rate. A frame corresponds to the data used to generate a given image or portion of an image associated with a particular type of data (e.g., B-mode data).

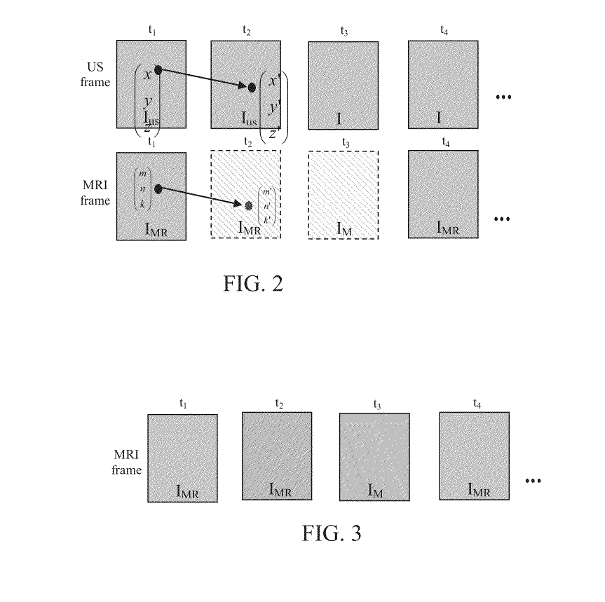

The acquisition is repeated at different times. The scanning of the region and detection of ultrasound data from the scanning are repeated. The repetition provides frames of ultrasound data representing the region at different times. For example, FIG. 2 shows the upper row including four frames of ultrasound data acquired for times t₁, t₂, t₃, and t₄. Different scans of the region are performed to acquire frames ultrasound data representing different times. Additional, different, or fewer frames may be acquired.

Any number of frames of the ultrasound data is acquired. For example, FIG. 2 shows acquisition of ultrasound data at four or more different times. The acquisition may be ongoing. A time sequence of frames is acquired from the ultrasound scanner.

Each frame represents the same or substantially same region. “Substantially” is used to account for unintended motion of the transducer relative to the patient or physiological motion of the patient. Frames representing overlapping but different regions may be used, such as where the transducer is translated and/or rotated.

Any rate of repetition may be used. For example, one frame representing an area or volume of the region is acquired each second. As another example, the rate is ten or more frames a second. The size of the scan region and beamformer capabilities of the ultrasound scanner may limit the frame rate.

In act 32, magnetic resonance (MR) data is acquired. The acquisition uses a MRI system. The MR data for an area or volume is acquired for a given time. The acquisition may be associated with a period. This period is treated as acquisition at a time. The data for a given time may represent instantaneous measurement or a temporal average. MR data representing the region at one time may be calculated from data also used to calculate the data for another time, such as in a moving window of data.

The MR data may be acquired at a same time as the acquisition of ultrasound data. Alternatively, the MR data is acquired before, after, or interleaved with the ultrasound data, such as for acquisition for the same time being relative to a cycle. MR data is acquired at substantially the same time as ultrasound data. “Substantially” accounts for the acquisition time of MRI being longer. For example, the MR acquisition of one frame occurs over a time in which three frames of ultrasound data are acquired. Since the frame of MR data represents the patient at a given time, the MR data may be acquired at a middle time or substantially same time as one of the ultrasound frames.

The MRI system and the ultrasound system are independent of each other. Alternatively, a combined system is provided, such as a transducer being mounted to a patient bed of the MRI system. Control, electronics, or processing may be shared or separate.

For magnetic resonance, the received MR data indicates projection intensities. Using tomography or other processing, the intensity of response from different locations is determined. Different pulse sequences may be used to detect different molecules and/or characteristics at the scan region.

MR anatomy data may be obtained. The MR anatomy data represents anatomy of the patient. The MR anatomy data represents a volume of the patient, such as representing voxels in an N×MxO arrangement. Alternatively, the MR anatomy data represents a plurality of separate slices (e.g., three parallel slices). In other embodiments, the MR anatomy data represents a single plane or area.

One or more transmitters produce an RF excitation field. A desired number of the transmitters are employed and connected through a corresponding number of transmit/receive switches to a corresponding number of coils in an RF coil array. The combined RF fields of the coil elements produce a prescribed B₁ field throughout the region of interest in the subject. The signal produced by the subject in response to the RF excitation field is picked up by the coil array and applied to the inputs of the set of receive channels. The received signal is at or around the Larmor frequency. When the B₁ field is not being produced, the transmit/receive switches connect each of the receive channels to the respective coil elements. Signals produced by the excited spins in the subject are picked up and separately processed as k-space and/or object space data.

Any MR procedure for acquiring data may be used. For example, T1-weighted or T2-weighted data is obtained. As another example, diffusion data is obtained.

The MR data are values for different locations of the region of the patient. The MR data is image data, k-space data, object-space data, or data at other stages of processing.

The region is of the same two or three-dimensional locations as for the ultrasound data. Alternatively, the region represented by the MR data overlaps with but is not identical to the region represented by the ultrasound data. The ultrasound and/or MR data may be converted to a same coordinate system, such as using data registration, fiducial-based transformation, or position sensors.

Frames of MR data are acquired for different times. Any rate of acquisition may be used. The rate for the MR data is less than the rate for the ultrasound data. The ultrasound image sequence is at a higher area or volume rate compared to the MRI sequence. In the example of FIG. 2, one frame of MR data is acquired for every three frames of ultrasound data. The MR data is acquired every third time (e.g., t₁ and t₄) and not acquired at other times (e.g., not acquired at times two and three (t₂ and t₃)). Other differences in rate may be provided.

Each frame represents the same or similar region of the patient. The MR data of a given frame represents the entire region of interest or field of view at any sample density. The scan region for MR may be the same or different but overlapping with the scan region of the ultrasound. The frames of MR data represent the sampled locations in an area or volume of the patient. The frames from different times may also represent different locations, such as associated with the patient moving relative to the MR system during the sequential scanning. The different frames may represent overlapping but different regions in these alternative embodiments.

The time axis may be generalized, such as each time representing a period. While the MR data and the ultrasound data may not represent the identical time, both may represent the patient region in a range of time. The overlapping range of time may be considered a same time. Any size range may be used, such as 2, 1, 0.1, 0.01, or 0.5 seconds.

In one embodiment, both types (e.g., MR and ultrasound) of data are acquired at the same or substantially same (e.g., with a same period or time range) absolute time. A timestamp of acquisition for each frame is used to temporally align the MR data with the ultrasound data in the time domain. Alternatively, the times for each frame of data may be relative to a trigger event (e.g., contrast agent destruction) or cycle. For example, the ultrasound data of one frame or volume may represent the patient at an R-wave of the heart cycle and be acquired at 1:23:45 pm. The MR data of one frame may represent the patient at the R-wave also, but be acquired at 1:23:50 pm. Both may be assigned time t₁ as the data represents the same time relative to the heart cycle.

Since frames of the ultrasound data are acquired with a greater frame rate in act 30 than the frames of MR data in act 32, more frames of ultrasound data than MR data over the same period result. FIG. 2 shows the set of four frames of high frame rate ultrasound data and the set of two frames of low frame rate MR data. As a result, frames of ultrasound data are available for times t₁, t₂, t₃ and t₄ whereas frames of MR data are available at times t₁ and t₄. The frame rate of the MR acquisition is less than the frame rate of the ultrasound acquisition. Any relative frame rates may be provided, such as the ultrasound frame rate being at least three times (e.g., 3 or 4 times) the MR frame rate.

The frames of MR and ultrasound data are acquired with the same spatial resolution. The scan settings for the ultrasound and MR systems are configured to acquire with the desired sampling resolution. Scan conversion, interpolation, extrapolation, decimation, filtering or other techniques may be used to create image or other MR or ultrasound data at the desired spatial resolution. In alternative embodiments, the MR and ultrasound data as acquired have different spatial resolution. The MR and ultrasound data are changed to a common resolution. Interpolation, extrapolation, filtering, decimation, down-sampling, up-sampling, or other conversion is provided. The MR data is converted to the resolution or sample grid of the ultrasound data, or vice versa. Both types of data may be converted to a third resolution of sample grid.

In act 40, the frames of ultrasound data are spatially registered with the frames of MR data. Since different systems are used to acquire the ultrasound and MR data, different coordinates, samples, pixels, or voxels may represent different locations in the patient. The frames are aligned so that the data of both types of data representing a given location of the patient are known. The spatial relationship of the coordinate systems is determined.

In one embodiment, the ultrasound system has a known spatial relationship with the MR system. For example, the transducer used for scanning with ultrasound is fixed or attached to the patient bed of the MR system. The spatial registration is based on the known spatial position of the different systems. Alternatively, one or more measurements and/or sensors are used to determine the relative position of the scanners. Calibration may be used. One system may be used to detect a component of another system for determining spatial position.

In other alternative embodiments, the ultrasound and MR data are used to spatially register. Using rigid or non-rigid transforms, the translation, rotation, and/or scaling of the ultrasound data to the MR data or vice versa is determined. The registration is based on the entire frames of data. Alternatively, a sub-set of the data, such as a region of interest is used. Multiple regions for the same frames may be used. In one example, the MR data is converted to emulate ultrasound data. Ultrasound data is synthesized from the MR data. The synthesized ultrasound data is registered with the acquired ultrasound data. Speckle or feature-based registration may be used. In other examples, features are extracted from both the MR and ultrasound data. The features are then used for registration. The registration uses correlation, minimum sum of absolute differences, or other measure of similarity to find the translation, rotation, and/or scale associated with the best match.

Motion between frames may be due to deformation and/or slip between regions. Scaling, translation and/or rotation may vary locally from region to region. The scaling in orthogonal directions at any point may be different, (e.g. stretch of incompressible tissue in the x-direction with x-scaling>1 and y-scaling<1). At a slip boundary, adjacent points on different sides of the slip boundary may move at different rates or in different directions. Fluid/tissue boundaries are a special case, but locally varying scaling, orthogonally varying scaling, translation and/or rotation may account for motion tracking in view of such boundaries.

The spatial registration based on data uses the frames from the same or substantially same time. For example, the frame of MR data at time t₁ is spatially registered with the ultrasound data from time t₁. Frames from different times may be spatially registered. One registration may be used (e.g., from one time) for all or multiple other frames. The relationship between the coordinates of the MR and ultrasound systems may not change during an imaging session. Alternatively, the spatial registration is ongoing or occurs periodically.

Once the ultrasound frames are temporally and/or spatially registered with the MR frames, the motion between times is determined in act 42. The frames of MR data are acquired for a sub-set of the times (e.g., t₁ and t₄). To create frames of MR data for other times (e.g., t₂ and t₃), the motion associated with those times is determined.

Since MR data is not available for these times, the motion is determined from the higher frame rate ultrasound data. A change in location over time for each of a plurality of locations represented in one or more frames of MR data is determined from ultrasound detected motion. The motion correction aligns locations represented by data over time with coordinates. Where motion causes a tissue location to shift relative to the scanning, the locations may be aligned by motion compensation.

The motion from one time to the next indicates a change in location. A given coordinate at time t₁ may represent one location in the patient, but that location may change by time t₂. For example, physiological motion or motion of the patient may result in change of location over time. In another example, the location of a part of a heart wall is at location 10, 12, 15 in the coordinate system of the ultrasound system at time one. The part of the heart wall moves relative to the ultrasound system by time two. Since the ultrasound system is at a same position relative to the patient, the part is at a different coordinate (e.g., 15, 18, 16) at time two. The same part of the heart is at different coordinates at different times.

Since different locations of the patient may change by different amounts, the determination of motion is localized. Any localization may be used, such as by location (e.g., single coordinate, pixel, or voxel) or by neighborhood (e.g., 5×5 or 3×3×3 region). Motion determination is performed for each location or neighborhood. In alternative embodiments, a single global motion for the entire frame is determined.

The motion is determined between temporally adjacent ultrasound frames. For example, FIG. 2 shows tracking a voxel location between times t₁ and t₂). The motion between the next two frames in a moving window may be determined. Alternatively, one frame is used as a reference for all motion, at least until another pair of both MR and ultrasound frames are acquired representing the region of the patient at a same or substantially same time.

A motion estimation algorithm is used to track the locations. In one embodiment, the estimation of motion to determine the change in location over time uses anatomy data. The anatomy is detected or extracted from the frames of ultrasound data. The features may be tissue boundaries, tissue regions, bone region, fluid region, air region, combinations thereof, or other feature. Motion estimation may operate more accurately with anatomy features. Alternatively, the motion estimation is performed with speckle information.

Any two or three-dimensional motion estimation or tracking may be used, such as rigid or non-rigid techniques. Local cross-correlation (LCC) cost function, minimum sum of absolute differences, or other measure of similarity is used for motion estimation. The frames of the sequence are compared for different possible motion. Different scaling, translations and/or rotations are tested. Global or local motion may be estimated. For each test, a level of similarity is calculated. The transition and rotation combination with the greatest level of similarity indicates the motion. Alternatively, velocity, acceleration or other Doppler parameter is used to determine the motion.

The motion is determined for the frames, locations, or neighborhoods. To determine motion associated with a single location, a kernel or neighborhood is identified. The similarity measure is performed for the kernel or neighborhood.

Any search pattern may be used. For example, a regular search pattern or exhaustive search pattern testing all combinations is used. In other embodiments, numerical optimization, course-to-fine searching, subset based searching, or use of decimated data is used to reduce computations.

The registration is along two or three-dimensions. Any combination of scaling, translation and rotation degrees of freedom may be used, such as 6 degrees (3 axes of rotation and 3 axes of translation).

The correlation may be based on all of the data in the sets or sub-sampled data. The correlation may be for data or for features. For example, a plurality of features is identified by the user or automatically by a processor. The data representing the features with or without surrounding data is used for the correlation. The features may be identified in one set (e.g., ultrasound) for matching with all of the data in another set, or features of one set may be matched to features of another set.

Where different amounts of change are provided for different neighborhoods of locations, the change for any location is based on the neighborhood for which the location is a member. In other embodiments, the change or motion is low pass filtered across locations.

For each coordinate (e.g., x, y, and z location), an amount of motion or change in location from one time to another is determined. The change may be the same or different for different locations. In the example of FIG. 2, the location x, y, z is shown shifting from the upper middle to the middle (x′, y′, z′) from time t₁ to time t₂. The different coordinates in the frames represent the same tissue location at different times. Other locations may have the same, similar, or different magnitudes and/or angles of change in location.

In act 44, a frame of constructed magnetic resonance data is constructed. The frame represents a time for which MR data is not acquired. In the example of FIG. 2, a frame of MR data is constructed for time t₂. The constructed frame represents the same region or at least part of the same region as the first frame, but accounts for motion over time (motion from time t₁ to time t₂).

The constructed frame of MR data is created using a frame of scan MR data and motion. Acquired (i.e., scan) MR data representing the region is adjusted to account for motion, creating the constructed frame. Since the motion is associated with a particular time, the constructed frame is for the particular time. In alternative embodiments, the constructed frame of MR data is created from two or more frames of scan MR data. For example, the frames of MR data acquired in act 32 immediately before and immediately after are used. Non-immediate frames in time may be used. Similarly, motion from one or more ultrasound frames may be used, such as determining a change in location using both forward and reverse motion estimation through the sequence.

The change in location indicates change in the locations of the MR data. Since the frames of ultrasound and MR data from the same or substantially same time are spatially registered, the ultrasound motion indicates the motion relevant for the MR data as well. Given the estimated motion parameters relating coordinates at times t₁ and t₂, the MR data point at time t₂ may be reconstructed even though MR data is not acquired in act 32 for time t₂. The reconstruction uses the MR data or value at t₁. The motion for the same location from the ultrasound data is applied to the MR data.

In FIG. 2, the motion or change in location represented by the vector in the ultrasound data is applied to the MR data. The scan acquired MR data is offset by the change. For example, the value of the MR data at location m, n, k in the frame at time t₁ is 64. Since this location moves by time t₂, the value of 64 is repositioned or offset based on the motion to location m′, n′, k′ for the constructed frame at time t₂. Intensities from the acquired or scan MR data of one frame are offset and assigned in the constructed frame of data based on the determined change in position.

Where a global change is used, the constructed frame may represent less than all of the area or volume represented by the scan frame. For localized changes, the constructed frame may include one or more holes or regions to which intensities where not mapped due to the motion. These holds or regions may be filed with interpolation, filtering, or other process. Due to rounding or inaccuracy, more than one value may be offset to a same coordinate. One value may be selected or the values may be averaged.

The constructed frame of constructed MR data may be filtered or processed. Any technique to limit artifacts from the construction may be used.

In act 46, the constructed frame of MR data is inserted into the sequence of frames of MR data. The frame rate of the temporal sequence of frames of MR data is increased. For example, the frame of constructed MR data representing time t₂ is inserted into the sequence with the frames of scan MR data representing times t₁ and t₄. The sequence including the constructed frame has a greater frame rate than the sequence with just the acquired frames. In alternative embodiments, the constructed frame is used for imaging without insertion into a sequence.

In act 48, an image is displayed. The image is based on the constructed frame. The frame of constructed MR data is mapped to display values, such as mapping to gray scale or color (e.g., RGB) values. The constructed MR data may be converted to a Cartesian coordinate or other format appropriate for a display device. Alternatively, the constructed MR data is in the format for display.

The generated image is displayed. For a two-dimensional area, the image represents an area of the patient. Where the MR data represents a volume, the image may be generated by surface, projection, or other volume rendering.

The image is output for display, such as outputting to a display. Alternatively, the images are output to a database, such as outputting for later retrieval.

The image is displayed as one image in a sequence of images. The sequence may include images generated from frames of scan MR data and images from frames of constructed MR data. The images are shown in order, such as to emulate real-time. Alternatively, the images are shown slowed down or sped up relative to real-time. The images are displayed during on-going acquisition in acts 30 and/or 32 or are shown after acquisition is complete. In alternative embodiments, the image is displayed without other images (e.g., no sequence).

As represented by the feedback from act 48 to act 42, the creation of additional frames of MR data may be repeated. The motion is determined in act 42 for different times, such as times for which MR data is not available but ultrasound data is available. For example, the motion is tracked from a reference frame (e.g., ultrasound frame at time t₁) or temporally adjacent frame (e.g., ultrasound frame at time t₂) to the frame at a next time (e.g., ultrasound frame at time t₃). The motion is tracked for any number of frames of ultrasound data acquired between any two temporally adjacent frames of scan MR data.

A frame of constructed MR data is created for each of the times for which motion is determined. The MR data is constructed from the acquired or constructed frame from which the motion is determined (e.g., from the frame at time t₁ or the frame at time t₂) for the desired time (e.g., to construct a frame for time t₃). The MR data used is any existing frame of MR data. The motion is from the reference frame to the desired time. In other embodiments, the motion between different pairs of frames may be combined to determine motion over a greater period, such as determining motion between time t₁ and time t₃ from tracked motion from time t₁ to time t₂ and from time t₂ to time t₃. The MR data of an existing frame, such as the acquired frame of time t₁ may be used to determine the constructed frame at time t₃ using the combined motion.

In one embodiment, a curve is fit to the motion over time. Using this curve, the motion at any given time may be estimated. Even for a time at which ultrasound data was not acquired, a frame of constructed MR data may be created. The motion from the fitted curve is used.

By repeating, reconstructed MR data at sequential times is created and inserted for display in a sequence. The repetition continues until a new frame of scan MR data becomes available. Once a new acquired frame of MR data is available, the new frame of scan MR data may be used for repetition to create later constructed frames for insertion.

The sequence of images may be displayed at the rate associated with ultrasound acquisition or another rate, such as a real-time rate of twenty or more frames a second. In the example of FIG. 2, two frames are created and inserted at times t₂ and t₃. As represented in FIG. 3, the MR sequence includes four frames, two of scan MR data and two of constructed MR data. Given the frames of scan MR data at times t₁ and t₄, the frame rate is increased by a factor of three. Greater or lesser increases in frame rate may be provided. The rate is greater than the MR acquisition rate.

Separate images for the separate modalities may also be provided. Ultrasound images may be displayed with the MR images. The images from the different modalities are fused or combined or are displayed separately.

As a result of inserting constructed frames, MRI occurs with a greater frame rate. More images are available for the same period. The sequence of images may provide signal-to-noise ratio and clarity of MRI, but with temporal resolution associated with ultrasound. The MRI rate is greater than the rate of acquisition of frames of scan MR data.

FIG. 4 shows a system 10 for ultrasound enhanced magnetic resonance imaging. The system 10 includes a memory 12, an MR system 14, an ultrasound system 16, a transducer 18, a processor 26, and a display 28. Additional, different, or fewer components may be provided. For example, a network or network connection is provided, such as for networking with a medical imaging network or data archival system. As another example, a user interface is provided. The MR system 14, transducer 18, and ultrasound system 16 may not be provided in some embodiments, such as where the ultrasound and MR data is acquired by transfer or from storage.

The processor 26 and display 28 are part of a medical imaging system, such as the diagnostic or therapy ultrasound system 16, MR system 14, or other system. Alternatively, the processor 26 and display 28 are part of an archival and/or image processing system, such as associated with a medical records database workstation or server. In other embodiments, the processor 26 and display 28 are a personal computer, such as desktop or laptop, a workstation, a server, a network, or combinations thereof.

The display 28 is a monitor, LCD, projector, plasma display, CRT, printer, or other now known or later developed devise for outputting visual information. The display 28 receives images, graphics, or other information from the processor 26, memory 12, MR system 14, or ultrasound system 16.

One or more images representing a region of the patient are displayed. At least some of the values of the image are determined, at least in part, from MR values. For example, a sequence of images is rendered from three-dimensional data sets (e.g., frames) of MR data. The sequence includes MR data acquired by the MR system 14 at one or more times and MR data constructed to represent other times. Ultrasound is used to enhance the MRI by providing tracking. The tracking is used to construct sets of MR data for times between acquisitions, increasing the MR frame rate. Two-dimensional images presenting a planar region of the patient may be displayed.

The magnetic resonance (MR) system 14 includes a cyromagnet, gradient coil, and body coil in an RF cabin, such as a room isolated by a Faraday cage. A tubular or laterally open examination subject bore encloses a field of view. A more open arrangement may be provided. A patient bed (e.g., a patient gurney or table) supports an examination subject, such as a patient with or without one or more local coils. The patient bed may be moved into the examination subject bore in order to generate images of the patient. Received signals may be transmitted by the local coil arrangement to the MR receiver via, for example, coaxial cable or radio link (e.g., via antennas) for localization.

Other parts of the MR system are provided within a same housing, within a same room (e.g., within the radio frequency cabin), within a same facility, or connected remotely. The other parts of the MR system may include cooling systems, pulse generation systems, image processing systems, and user interface systems. Any now known or later developed MR imaging system may be used. The location of the different components of the MR system 14 is within or outside the RF cabin, such as the image processing, tomography, power generation, cooling systems, and user interface components being outside the RF cabin. Power cables, cooling lines, and communication cables connect the pulse generation, magnet control, and detection systems within the RF cabin with the components outside the RF cabin through a filter plate.

The MR system 14 is configured by software, hardware, or both to acquire data representing a plane or volume in the patient. In order to examine the patient, different magnetic fields are temporally and spatially coordinated with one another for application to the patient. The cyromagnet generates a strong static main magnetic field B₀ in the range of, for example, 0.2 Tesla to 3 Tesla or more. The main magnetic field B₀ is approximately homogeneous in the field of view.

The nuclear spins of atomic nuclei of the patient are excited via magnetic radio-frequency excitation pulses that are transmitted via a radio-frequency antenna, such as a whole body coil and/or a local coil. Radio-frequency excitation pulses are generated, for example, by a pulse generation unit controlled by a pulse sequence control unit. After being amplified using a radio-frequency amplifier, the radio-frequency excitation pulses are routed to the body coil and/or local coils. The body coil is a single-part or includes multiple coils. The signals are at a given frequency band. For example, the MR frequency for a 3 Tesla system is about 123 MHz+/−500 KHz. Different center frequencies and/or bandwidths may be used.

The gradient coils radiate magnetic gradient fields in the course of a measurement in order to produce selective layer excitation and for spatial encoding of the measurement signal. The gradient coils are controlled by a gradient coil control unit that, like the pulse generation unit, is connected to the pulse sequence control unit.

The signals emitted by the excited nuclear spins are received by the local coil and/or body coil. In some MR tomography procedures, images having a high signal-to-noise ratio (SNR) may be recorded using local coil arrangements (e.g., loops, local coils). The local coil arrangements (e.g., antenna systems) are disposed in the immediate vicinity of the examination subject on (anterior), under (posterior), or in the patient. The received signals are amplified by associated radio-frequency preamplifiers, transmitted in analog or digitized form, and processed further by the MR receiver. Digitization occurs at the local coils or at the MR receiver.

The measured data is stored in digitized form as complex numeric values in a k-space matrix. A one or multidimensional Fourier transform reconstructs the object or patient space from the k-space matrix data.

The MR system 14 may be configured to acquire different types of data. For example, the MR data is intensities representing the anatomy of the patient. The MR data represents the response to the magnetic fields and radio-frequency pulses of tissue. Any tissue may be represented, such as soft tissue, bone, or blood. The MR system 14 may be configured for acquiring specialized functional or anatomic information. For example, T1-weighted, diffusion, or T2-weighted MR data is acquired.

The MR system 14 scans the patient over time. A sequence of frames of MR data is acquired. Any rate of acquisition of the MR frames may be used. The rate may vary over time. The acquired frames represent the patient at different times. The MR values may be associated with better signal-to-noise ratio, but less rapid frame rate than ultrasound values acquired using the ultrasound system 16.

The ultrasound system 16 is any now known or later developed ultrasound imaging system. For example, the ultrasound system 16 includes the transducer 18 for converting between acoustic and electrical energies. Transmit and receive beamformers relatively delay and apodize signals for different elements of the transducer 18. B-mode, Doppler, or other detection is performed on the beamformed signals. A scan converter, memory, three-dimensional imaging processor, and/or other components may be provided.

The transducer 18 is a one-, two-, or multi-dimensional array of piezoelectric or capacitive membrane elements. In one embodiment, the transducer 18 is a handheld or machine held transducer for positioning against and outside of the patient. In another embodiment, the transducer 18 is part of a probe for use within the patient, such as a transesophageal probe. For example, the transducer 18 is a one-dimensional array of elements within or on a catheter used for intervention or a different purpose. In yet another embodiment, the transducer is positioned in a patient bed of the MR system or by a robot for use on the patient while in the MR bore for scanning. Any electronics in the transducer 18 are shielded and/or have blocking filters to limit emissions at frequencies used by the MR system 14.

The ultrasound data is output in a polar coordinate or scan converted Cartesian coordinate format. Acoustic energy is used to scan a plane and/or volume. For example, a volume is scanned by sequentially scanning a plurality of adjacent planes. Any format or scan technique may be used. The scanned volume may intersect or include all of the patient volume.

The ultrasound system 16 is configured by software, hardware, or both to acquire sets of ultrasound data representing the patient at different times. A sequence of frames is acquired. The scan or frame rate may be greater than the frame rate of the MR system 14. For example, the ultrasound frame rate is at least twice the MR frame rate for scanning a same or substantially same field of view with a same or substantially same spatial resolution.

The memory 12 is a graphics processing memory, video random access memory, random access memory, system memory, cache memory, hard drive, optical media, magnetic media, flash drive, buffer, database, combinations thereof, or other now known or later developed memory device for storing data or video information. The memory 12 is part of an imaging system, part of a computer associated with the processor 26, part of a database, part of another system, or a standalone device.

The memory 12 stores datasets (e.g., frames) each representing a three-dimensional patient volume or a two-dimensional patient area. The patient volume or area is a region of the patient, such as a region within the chest, abdomen, leg, head, arm, or combinations thereof. The patient area or volume is a region scanned by the MR system 14 and the ultrasound system 16.

Any type of data may be stored, such as medical image data (e.g., ultrasound and MR anatomy data). The data represents the patient over time, such as prior to or during treatment or other procedure.

The stored data is interpolated or converted to an evenly spaced two or three-dimensional grid or is in a scan format. The data for different modalities may be transformed to be on a same grid or format. The data from different modalities may be spatially registered.

The memory 12 or other memory is a non-transitory computer readable storage medium storing data representing instructions executable by the programmed processor 26 for ultrasound enhanced magnetic resonance imaging. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The processor 26 is a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device for determining motion from ultrasound data and using the motion to construct additional frame of MR data from MR data representing the patient at a different time. The processor 26 is a single device or multiple devices operating in serial, parallel, or separately. The processor 26 may be a main processor of a computer, such as a laptop or desktop computer, or may be a processor for handling tasks in a larger system, such as the MR or ultrasound systems 14, 16. The processor 26 is configured by software and/or hardware.

The processor 26 is configured to determine spatial offsets over time from the ultrasound data. The change in location over time of tissue, structure, or other anatomy of the patient is tracked using ultrasound. Spatial offsets for different locations represented in each of the frames are determined. Using the tracking, the location of anatomy, tissue, or other structure at a given time is determined. Ultrasound data from one time is compared with ultrasound data for the desired time.

The change in location of the tissue is used to increase a frame rate of MR images. The spatial offsets are applied to the MR data. MR data for a given time is transformed by the spatial offsets. The coordinates of the MR data are changed as indicated by the spatial offsets so that the same values for given tissue or structure are located at the appropriate coordinates given the time (i.e., motion to get to the time). The values of the MR data are maintained, but the locations associated for the values are shifted to account for any tissue or structure motion.

The processor 26 may create any number of sets of MR data. Using the tracking, the motion between a time represented by an available set of MR data and a time for which an MR set is to be constructed is determined. The available MR data is then used, with the spatial offsets, to create another set of MR data representing the patient at that different time.

The created sets are interleaved with the acquired sets of MR data. The interleaved sequence is used to generate images. The images may be generated at a greater frame rate than the MR scanner acquires frames or images.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for ultrasound enhanced magnetic resonance imaging, the method comprising:
acquiring ultrasound data representing a region of a patient at a first rate, a first frame of the ultrasound data for the region being acquired at a first time and a second frame of the ultrasound data for the region being acquired at a second time after the first time;
acquiring scan magnetic resonance data representing the region of the patient at a second rate less than the first rate, a first frame of the scan magnetic resonance data for the region being acquired at substantially the first time and a second frame of the scan magnetic resonance data for the region being acquired at a third time after the first and second times;
determining motion from the first time to the second time from the first and second frames of the ultrasound data;
constructing a third frame of constructed magnetic resonance data from the first frame of the scan magnetic resonance data and the motion, the third frame representing the region at the second time; and
displaying an image as a function of the third frame of the constructed magnetic resonance data.

2. The method of claim 1 wherein acquiring the ultrasound data comprises acquiring with the first rate being at least three times the second rate.

3. The method of claim 1 wherein acquiring the ultrasound data comprises acquiring B-mode data, and wherein acquiring the scan magnetic resonance data comprises acquiring image data.

4. The method of claim 1 wherein acquiring the scan magnetic resonance data comprises acquiring in response to a sequence of radio frequency pulses transmitted to the region with a magnetic resonance imaging system.

5. The method of claim 1 wherein the region comprises a volume, and wherein acquiring the ultrasound data and the scan magnetic resonance data comprise acquiring the first and second frames of the ultrasound data and the scan magnetic resonance data as each representing the entire volume.

6. The method of claim 1 wherein determining the motion comprises calculating a scale, translation, rotation, or combinations thereof of the second frame of ultrasound data relative to the first frame of ultrasound data with a highest correlation, the motion being the scale, translation, rotation, or combinations thereof.

7. The method of claim 1 wherein determining the motion comprises determining the motion for a first location in the region and repeating the determining for other locations in the region, the motions for the other locations being the same or different than the motion for the first location, and wherein constructing comprises offsetting the scan magnetic resonance data of the first frame for the first and other locations with the respective motions.

8. The method of claim 1 wherein constructing the third frame comprises assigning locations of the scan magnetic resonance data of the first frame in the third frame based on the motion.

9. The method of claim 1 wherein displaying comprises displaying the image as one in a sequence, the sequence being of magnetic resonance images at the first rate, the first rate greater than the second rate of acquisition of the scan magnetic resonance data.

10. The method of claim 1 further comprising:
spatially registering the first frame of ultrasound data with the first frame of the scan magnetic resonance data;
wherein constructing comprises constructing as a function of the spatially registering.

11. The method of claim 1 further comprising:
repeating the determining, the constructing, and the displaying for subsequent times for which any frame of scan magnetic resonance data is unavailable.

12. In a non-transitory computer readable storage medium having stored therein data representing instructions executable by a programmed processor for ultrasound enhanced magnetic resonance imaging, the storage medium comprising instructions for:
generating, by magnetic resonance scanning, a temporal sequence comprising sets of first magnetic resonance data;
determining, with ultrasound, a change in location over time for each of a plurality of locations represented in at least a first set of the sets of magnetic resonance data;
creating additional sets of magnetic resonance data from one or more of the sets of first magnetic resonance data as a function of the changes in locations; and
inserting the additional sets into the temporal sequence such that the temporal sequence of the sets and additional sets has a greater frame rate than the sets generated by magnetic resonance scanning.

13. The non-transitory computer readable storage medium of claim 12 wherein generating comprises acquiring the sets of first magnetic resonance data in response to a sequence of radio frequency pulses transmitted to a patient with a magnetic resonance imaging system

14. The non-transitory computer readable storage medium of claim 12 wherein determining comprises calculating a scale, translation, rotation, or combinations thereof of the second frame of ultrasound data relative to the first frame of ultrasound data as a function of similarity, the change being the scale, translation, rotation, or combinations thereof.

15. The non-transitory computer readable storage medium of claim 12 wherein creating comprises assigning intensities of the first magnetic resonance data of the first set to a first additional set with the locations based on the changes.

16. The non-transitory computer readable storage medium of claim 12 wherein inserting comprises inserting at least two of the additional sets for every one of the sets of first magnetic resonance data.

17. The non-transitory computer readable storage medium of claim 12 further comprising displaying magnetic resonance images at the greater frame rate, the magnetic resonance images each corresponding to one of the sets or additional sets of the temporal sequence.

18. A system for ultrasound enhanced magnetic resonance imaging, the system comprising:
a magnetic resonance (MR) system configured to provide a first sequence of frames of MR data;
an ultrasound system configured to provide a second sequence of frames of ultrasound data; and
a processor configured to determine spatial offsets over time from the ultrasound data and to increase a frame rate of MR images applying the spatial offsets to the MR data.

19. The system of claim 18 further comprising a display operable to display the MR images at the frame rate, the frame rate being greater than the scan rate of the MR system.

20. The system of claim 18 wherein the processor is configured to determine the spatial offsets for different locations represented in each of the frames or volumes, the spatial offsets determined based on tracking, and wherein the processor is configured to create interleaved frames or volumes from the frames or volumes of the MR data by shifting the locations represented by the MR data based on the spatial offsets from the ultrasound data.
